(19) United States
(12) Patent Application Publication
(10) Pub. No.: US 2026/0048167 A1
(43) Pub. Date: Feb. 19, 2026

(54) MEDICAL WASTE STERILIZER

(71) Applicant: MECOBI CO.,LTD., Wonju-si Gangwon-do (KR)

(72) Inventors: Ir Win SOHN, Namyangju-si Gyeonggi-do (KR); Yong Gu KIM, Wonju si Gangwon-do (KR); Yong Beom PARK, Chungcheongnam-do (KR)

(73) Assignee: MECOBI CO.,LTD., Wonju-si Gangwon-do (KR)

(21) Appl. No.: 18/996,196

(22) PCT Filed: Jul. 25, 2022
(86) PCT No.: PCT/KR2022/010921

§ 371 Date: Jan. 17, 2025

(51) Int. Cl. A61L 11/00; A61L 2/18; A61L 2/22; A61L 2/24; B02C 23/36; B09B 3/35; B09B 3/38; B09B 3/70; B09B 101/65

(52) U.S. Cl. CPC A61L 11/00; A61L 2/18; A61L 2/22; A61L 2/24; B02C 23/36; B09B 3/35; B09B 3/38; B09B 3/70; A61L 2202/121; A61L 2202/122; A61L 2202/14; A61L 2202/15; B09B 2101/65

(57) ABSTRACT

The present invention relates to a medical waste sterilizer, and the medical waste sterilizer according to the present invention comprises two sterilization vessels which are slidable in the left-and right direction, whereby a waste disposal operation can be performed in one sterilization vessel while a sterilization operation is performed in the other sterilization vessel. Accordingly, there is an advantage in that the waste disposal capacity can be enhanced and the wait time can be reduced.

### TECHNICAL FIELD

The present invention relates to a medical waste sterilizer. More specifically, the present invention relates to a medical waste sterilizer capable of immediately disposing contaminated bacteria, etc. generated in medical settings within a short period of time.

### BACKGROUND ART

Wastes generated in hospitals and various medical settings include gauzes contaminated with blood, IV lines, needles, medical instruments, cotton wool, bandages, diapers, human body extracts, syringes, injection needles, thermometers, test tubes, various diagnostic instruments, analysis devices, and medication containers, among others. These wastes, which are referred to as medical wastes, may be contaminated with pathogenic bacteria, etc. that can affect environments and human bodies.

The contaminated wastes, which are classified as special medical wastes according to relevant laws and regulations, are segregated from general wastes and separately discharged, collected and incinerated. It is because they can cause critical issues to human bodies and environments in case they are discharged together with general wastes. However, the above process involves an increase in time, workload and logistical complexity relating to generation, segregation, collection, and incineration of contaminated wastes. It also involves an elevated risk of outward propagation of pathogenic bacteria in medical wastes. Another issue is a significant increase in management cost of contaminated wastes compared to that of general wastes. Additionally, there exist concerns about environmental pollution due to toxic gases possibly generated during incineration.

On the other hand, it is possible to dispose of medical wastes as general wastes if pathogenic bacteria are sterilized and removed from medical wastes before discharge, ensuring environmental safety and reducing processing costs. In previously known sterilizers, medical wastes are shred into small pieces before sterilization to efficiently operate sterilization and reduce waste volume. Traditional medical waste sterilizers employ steam or radiofrequency methods for sterilization. Korean Registered Patent No. 10-2126463 relates to a medical waste disposal operation device which utilizes steam and radiofrequency. It comprises a sterilization unit that sterilizes the shredded wastes by steam of over 160° C. and radiofrequency generated inside the waste shredder, and a transfer sterilization unit that transports and sterilizes the shredded wastes using hot air and radiofrequency during transfer from the waste shredder. Additionally, Korean Registered Patent No. 10-2058433 relates to a heat treatment device capable of disposing medical wastes, equipped with a reaction chamber for heat treatment and a steam supply unit supplying high-temperature and high-pressure steam to the target material.

However, these devices, being facility installations rather than portable equipment, require large installation areas and incur high installation costs and energy usage expenses. Hence, they pose a disadvantage for adoption in hospitals and medical settings that generate medical wastes, due to the difficulty in accommodating them.

This underscores the need for compact medical waste sterilizers that can reduce installation and energy costs while immediately processing medical wastes generated at medical settings within a short period of time.

### PRIOR ART

### Patent Literature

(Patent Literature 1) Korean Registered Patent No. 10-2126463
(Patent Literature 2) Korean Registered Patent No. 10-2058433

### DETAILED DESCRIPTION

### Technical Objective

The present invention is designed to address the conventional problems mentioned above. The purpose of the present invention is to provide a medical waste sterilizer that can be manufactured in a size suitable for installation in general hospitals or medical settings, which can sterilize contaminated bacteria, etc. within medical wastes in a short period of time and discharge the same as general wastes.

Another purpose of the present invention is to offer a medical waste sterilizer that facilitates washing, sterilizing, and dehydration of medical wastes after they are finely shredded.

Another purpose of the present invention is to provide a medical waste sterilizer that enhances the waste disposal capacity by simultaneously performing sterilization of medical wastes and waste disposal operations.

### Means for Solving the Problems

To achieve the above objectives, the medical waste sterilizer according to the present invention comprises two sterilization vessels which are slidable in the left-and-right direction, whereby a waste disposal operation is performed in one sterilization vessel while a sterilization operation is performed in the other sterilization vessel in an automatic manner. Specifically, it requires time for the sterilizing solution to react with the medical wastes, and in case that there is only one sterilization vessel, waste disposal is ceased until the sterilization operation is completed. To address this disadvantage, the medical waste sterilizer according to the present invention comprises two sterilization vessels which are slidable in the left-and-right direction, whereby a waste disposal operation can be performed in one sterilization vessel while a sterilization operation is performed in the other sterilization vessel, providing advantages of the reduced wait time and the enhanced waste disposal capacity.

In an embodiment of the present invention, the medical waste sterilizer is equipped with a rotating agitation device for sterilization vessels that rotates and agitates diluted sterilizing solution and shredded wastes, which enables improvement of sterilization efficiency, sterilization of the contaminants on the other side of the wastes, and reduction of the final moisture content in the wastes by dehydration after the sterilization operation is completed.

In an embodiment of the present invention, the medical waste sterilizer may be equipped with a tilting drive unit for sterilization vessels that tilts the sterilization vessels after the sterilization operation to facilitate easy discharge of wastes from the front.

In an embodiment of the present invention, the medical waste sterilizer may be equipped with an automatic waste feeder with conveyor for heavy or bulky waste, which automatically transports the wastes placed on the conveyor to the inlet at the rear of the top of the sterilizer, where it is fed into the shredder.

In an embodiment of the present invention, the sterilizing solution used in the medical waste sterilizer may be a known, strongly acidic solution, in which a neutralizer can be added after sterilization is completed so that the solution can be discharged with a higher pH.

In an embodiment of the present invention, the medical waste sterilizer may be equipped with a damper at the lower part of the lower hopper of the shredder to prevent the wastes remaining inside the shredder from falling outside the sterilization vessel when the sterilization vessel is being slid in the left-and-right direction after the operation of the shredder is complete.

In an embodiment of the present invention, the medical waste sterilizer employs a high-pressure metering pump to sequentially spray sterilizing solution and water from spray nozzles, allowing them to be mixed and diluted as needed. In this way, the need for a large dilution tank required for pre-dilution of the sterilizing solution can be eliminated, enabling the downsizing of the equipment.

In an embodiment of the present invention, the medical waste sterilizer may be equipped with a waste strainer with a mesh structure inside the sterilization vessel to facilitate easy separation and waste disposal.

In an embodiment of the present invention, the medical waste sterilizer may be equipped with a barcode reader for the wastes on the equipment, which is connected to the hospital's server to track and manage whether the wastes have been properly processed by transmitting information related to disposal location, date of generation, disposal weight, date of disposal, etc.

In an embodiment of the present invention, the medical waste sterilizer may be equipped with an automatic waste discharge device for sterilization vessels, which automatically discharges the sterilized wastes from within the sterilization vessel by raising the lower lift using a drive screw for lift.

In an embodiment of the present invention, the medical waste sterilizer, unlike methods that utilize steam or microwaves for sterilization, operates independently without the need for external devices. Further, it can be installed in various locations without specific constraints because it can function without requiring additional facilities. Furthermore, it can save energy that would otherwise be required for steam or microwaves generation.

In an embodiment of the present invention, the medical waste sterilizer allows for easy cleaning and sterilization of contaminants inside the shredder, which is a task challenging to perform with steam and microwaves methods.

### Working Effect of the Invention

The medical waste sterilizer of the present invention performs sterilization through chemical methods and does not require a separate dilution tank, allowing for downsizing of the equipment. It can also be easily installed in existing hospitals or medical settings to instantly treat contaminated bacteria, etc. generated in medical settings in a short time and discharge them as general wastes. Consequently, it prevents the spread of hospital-acquired pathogens from medical wastes to external and provides a desirable effect of significantly reducing energy consumption compared to conventional equipment. Furthermore, by simultaneously performing sterilization and waste disposal operations, it provides effects of enhancing the waste disposal capacity and saving wait time for sterilization.

### BRIEF DESCRIPTION OF DRAWINGS

FIG. 1 shows a first front view of the medical waste sterilizer in one embodiment of the present invention.

FIG. 2 shows a second front view of the medical waste sterilizer in one embodiment of the present invention.

FIG. 3 shows a first side view of the medical waste sterilizer in one embodiment of the present invention.

FIG. 4 shows a second side view of the medical waste sterilizer in one embodiment of the present invention.

FIG. 5 shows a rear view and conveyor of the medical waste sterilizer in one embodiment of the present invention.

FIG. 6 shows an automatic discharge device for sterilization vessels of the medical waste sterilizer in one embodiment of the present invention.

### MODE FOR CARRYING OUT THE INVENTION

Hereinafter, preferred embodiments of the present invention will be described in detail with reference to the accompanying drawings. However, it should be noted that the following embodiments are provided to fully disclose the invention and sufficiently convey its configuration and technical features to those skilled in the art, and the present invention is not limited to the embodiments disclosed herein and may be implemented in various other forms.

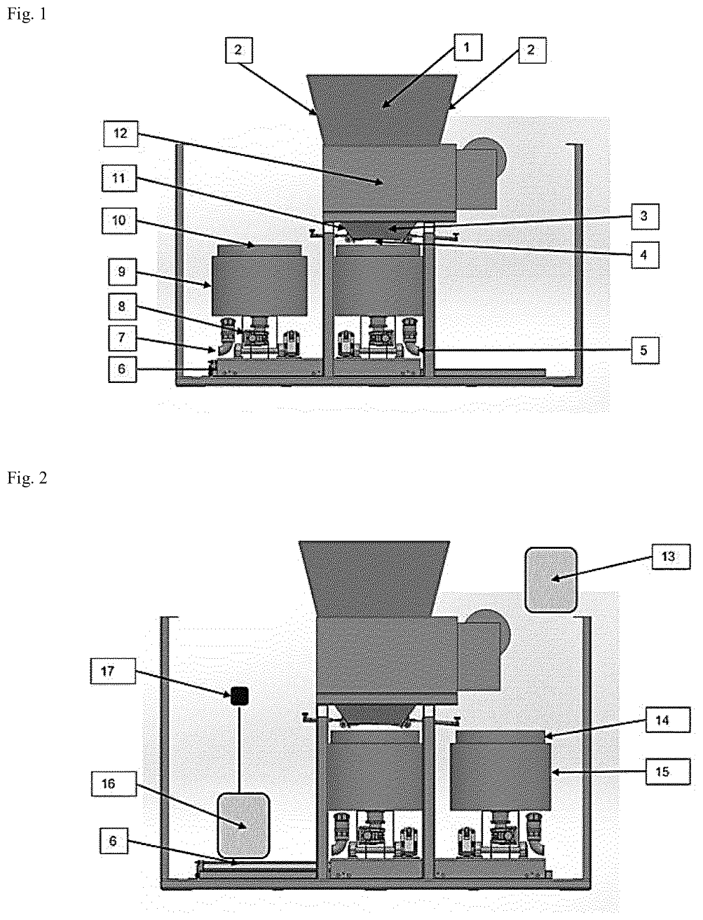

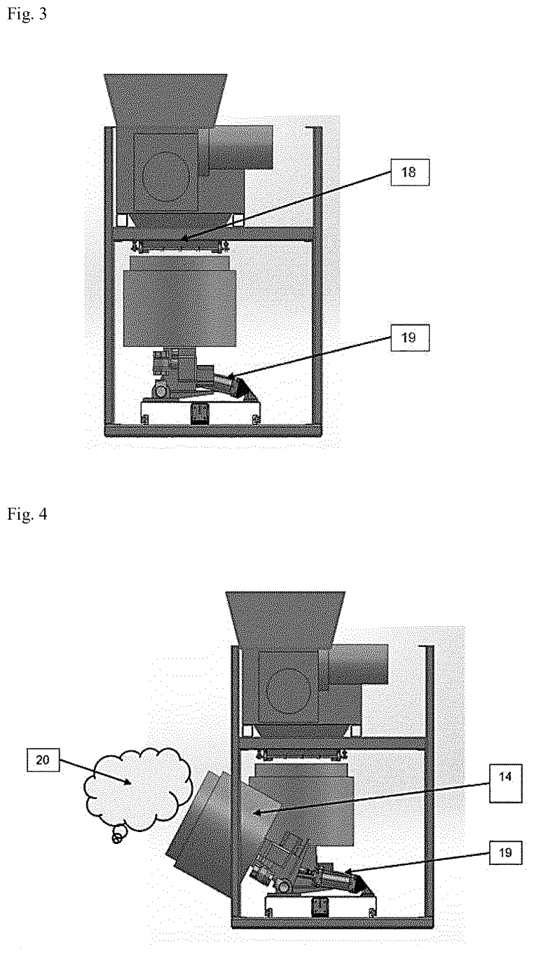

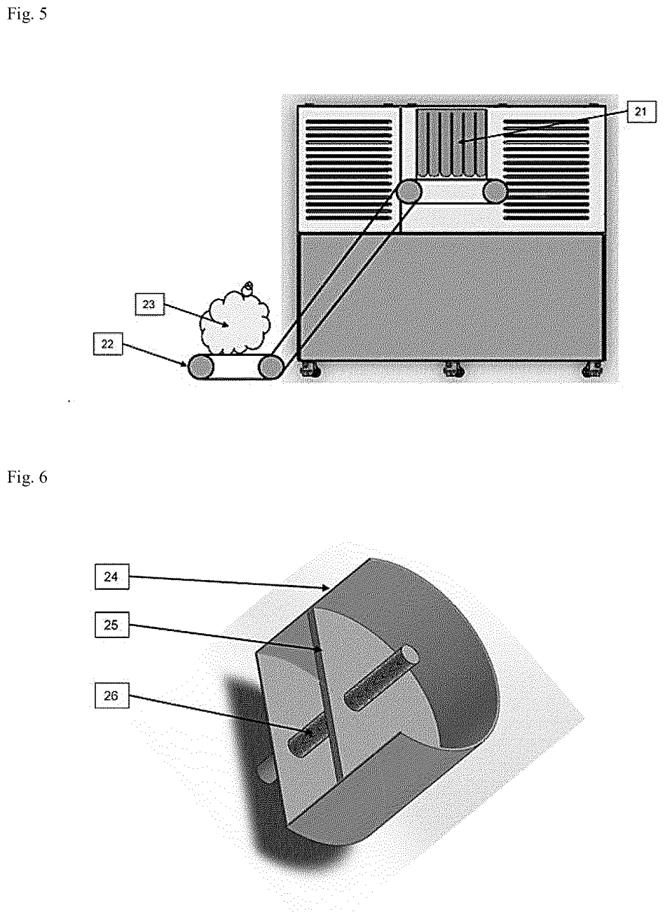

FIG. 1 and FIG. 2 show a front view illustrating the configuration of the medical waste sterilizer in one embodiment of the present invention., and FIG. 3 and FIG. 4 show a side view of the medical waste sterilizer in one embodiment of the present invention. FIG. 5 shows a rear view and a conveyor of the medical waste sterilizer in one embodiment of the present invention, and FIG. 6 shows an automatic discharge device for sterilization vessels of the medical waste sterilizer in one embodiment of the present invention.

Hereinafter, the operation of the medical waste sterilizer according to the present invention will be described with reference to FIG. 1 to FIG. 6.
Step 1. When an operator deposits medical wastes into the upper hopper (1), the shredder (12) starts operating to shred the wastes. Subsequently, the lower damper (4) opens, and a diluted sterilization solution is sprayed through the upper spray nozzle (2) and the lower spray nozzle (11) installed in the upper hopper (1) and the lower hopper (3), respectively. The waste shreds are collected in the right-side sterilization inner vessel (14) along with the sterilization diluted solution.
Step 2. A detection device (not shown) installed in the right-side sterilization inner vessel (14) detects the amounts of shreds, and when the shreds accumulate to a certain height, the shredder (12) shreds only the shreds remaining inside and stops. Subsequently, sterilization solution is sprayed through the upper spray nozzle (2) and the lower spray nozzle (11), sterilizing and cleaning the contaminants in the upper hopper (1) and inside the shredder (12).
Step 3. A metering pump (17) dispenses a certain amount of sterilizing solution through the upper spray nozzle (2) and the lower spray nozzle (11). Subsequently, only water is sprayed, resulting in the sterilization solution in the upper hopper (1) and the shredder (12) being cleaned together.
Step 4. The detection device detects the water level of the sterilization vessel (24), and when the water level is detected at a set height, it stops water injection and closes the damper (4) at the lower part of the lower hopper (3) to prevent further shreds from falling.
Step 5. The right-side sterilization inner vessel (14) performs a sterilization operation as it rotates and agitates.
Step 6. Once the sterilization operation for the predetermined duration is completed in the right-side sterilization inner vessel (14), the left-and-right sliding device for sterilization vessels (6) simultaneously moves the right-side sterilization outer vessel (15) and the left-side sterilization outer vessel (9) to the right (see FIG. 2). Subsequently, the same process as Steps 1-5 is repeated to supply shredded materials and sterilizing solution to the left-side sterilization inner vessel (10).
Step 7. In the right-side sterilization inner vessel (14), a neutralizer is introduced and agitation is carried out to lower the pH concentration of the sterilizing solution.
Step 8. After agitation in the right-side sterilization inner vessel (14) is completed, the discharge valve at the lower part of the sterilization vessel (24) is opened to discharge water, and dehydration is then carried out as the sterilization vessel (24) rotates.
Step 9. Once dehydration is completed, tilting drive unit for sterilization vessel (19) tilts the sterilization vessel (24), allowing the wastes in the waste strainer (20) to be discharged.
Step 10. After the wastes are discharged, the tilting drive unit for sterilization vessel (19) operates to return the sterilization vessel (24) to the original position. Subsequently, when the shredding in the left-side sterilization outer vessel (9) is completed, left-and-right sliding device for sterilization vessel (6) moves the right-side sterilization outer vessel (15) and the left-side sterilization outer vessel (9) to the left (see FIG. 1).
Step 11. The same operations as in Steps 1-5 are performed in the right-side sterilization inner vessel (14), and the same operations as in Steps 7-9 are performed in the left-side sterilization inner vessel (10).
Step 12. The entire process of Steps 1-11 is repeated until the medical wastes are depleted.

The present invention is not limited to the specific preferred embodiments described above, and it is apparent to those skilled in the art that various modifications are possible without departing from the technical gist of the present invention as recited in the claims, and such modifications are also included within the claims of the invention.

### INDUSTRIAL APPLICABILITY

The medical waste sterilizer of the present invention performs sterilization through chemical methods and does not require a separate dilution tank, allowing for downsizing of the equipment. It can be easily installed in existing hospitals or medical settings to instantly treat contaminated bacteria, etc. generated in medical settings in a short time and discharges them as general wastes. Consequently, it prevents the spread of hospital-acquired pathogens from medical wastes to external and significantly reduces energy consumption compared to conventional equipment. Furthermore, by simultaneously performing sterilization and waste disposal operations, it enhances the waste disposal capacity and saves wait time for sterilization, making it industrially viable.

### DESCRIPTION OF REFERENCE NUMERALS

1: upper hopper
2: upper hopper spray nozzle
3: lower hopper
4: damper
5: right-side water outlet
6: left-and-right sliding device for sterilization vessel
7: left-side water outlet
8: rotating drive unit for sterilization vessel
9: left-side sterilization outer vessel
10: left-side sterilization inner vessel
11: lower hopper spray nozzle
12: shredder
13: neutralizer tank
14: right-side sterilization inner vessel
15: right-side sterilization outer vessel
16: sterilizing solution tank
17: metering pump
18: neutralizer nozzle
19: tilting drive unit for sterilization vessel
20: waste strainer
21: rear waste inlet
22: automatic feeder with conveyor
23: garbage bag
24: sterilization vessel
25: waste lift plate
26: drive screw for lift plate

1. A medical waste sterilizer, comprising:
a shredder for shredding medical wastes that requires a sterilization operation;
spray nozzles capable of spraying acidic sterilizing solution or water onto the medical wastes being shredded in the shredder; and
a first sterilization vessel and a second sterilization vessel for a sterilization operation of the medical wastes sprayed with the sterilizing solution and for a sequential waste disposal operation after the sterilization operation,
wherein while a sterilization operation of medical wastes is in progress in one sterilization vessel, a waste disposal operation of sterilized medical wastes is performed in the other sterilization vessel.

2. The medical waste sterilizer of claim 1, further comprising:
a lower damper (4) located at the lower part of the shredder (12),
wherein the lower damper (4), when opened, directs the medical wastes shredded by the shredder (12) into the interior of either the first sterilization vessel or the second sterilization vessel, along with water, sterilizing solution, or a mixture of water and sterilizing solution sprayed from spray nozzles.

3. The medical waste sterilizer of claim 2, further comprising:
a detection device for detecting a water level in the first sterilization vessel and the second sterilization vessel,
wherein the lower damper (4) can be opened or closed by the detection device, and
wherein the detection device can cease the spraying of water or sterilizing solution from the spray nozzles and close the lower damper (4) when the detection device detects a water level at a set height.

4. The medical waste sterilizer of claim 3,
wherein the first sterilization vessel and the second sterilization vessel are each capable of rotating agitation, and
wherein, after the lower damper (4) is closed, the first or the second sterilization vessel comprising the medical waste shreds together with water or sterilizing solution performs the sterilization operation while rotating agitation.

5. The medical waste sterilizer of claim 4,
wherein the first and second sterilization vessels further comprise at least one of:
a neutralizer nozzle (18) for introducing a neutralizer to reduce a pH concentration of the sterilizing solution;
a discharge valve located at a lower part of the sterilization vessel, which can be opened to discharge water after the neutralization reaction;
a waste strainer (20); and
a tilting drive unit for sterilization vessel (19), which can tilt the sterilization vessel to discharge the wastes in the waste strainer (20) and then return the sterilization vessel to the original position after water is discharged.

6. The medical waste sterilizer of claim 5, further comprising:
an upper hopper (1) located at an upper part of the shredder (12); and
a lower hopper (3) located at the lower part of the shredder (12),
wherein medical wastes requiring the sterilization operation are introduced into the shredder (12) via the upper hopper (1),
the lower damper (4) is located at the lower part of the lower hopper (3),
and the spray nozzles comprise an upper spray nozzle (2) installed in the upper hopper (1) and a lower spray nozzle (11) installed in a lower hopper (3).

7. The medical waste sterilizer of claim 6, further comprising:
a metering pump (17) to regulate an amount of water or sterilizing solution sprayed from the spray nozzles,
wherein the metering pump sequentially sprays sterilizing solution and water from the spray nozzles, allowing them to be mixed and diluted.

8. The medical waste sterilizer of claim 7,
wherein an automatic medical waste discharge device is equipped with a lift plate (25) and a drive screw for lift plate (26) inside the first sterilization vessel and the second sterilization vessel.

9. The medical waste sterilizer of claim 8, comprising:
a waste inlet (21); and
an automatic feeder with conveyor (22),
and further comprising:
an automatic medical waste feeder with conveyor capable of automatically transporting medical wastes and delivering them to the shredder (12) in the medical waste sterilizer.

10. The medical waste sterilizer of claim 1, further comprising:
a left-and-right sliding device for sterilization vessels (6) capable of simultaneously sliding the first sterilization vessel and the second sterilization vessel in the left-and-right direction,
wherein the left-and-right sliding device for sterilization vessels (6) slides the first sterilization vessel or the second sterilization vessel, in which the sterilization operation is completed, to the left or right in order to perform a waste disposal operation in the vessel where sterilization operation is completed, and
simultaneously to perform a new sterilization operation in the other sterilization vessel.

11. The medical waste sterilizer of claim 1, further comprising a barcode reader connected to a server,
which is capable of reading a barcode attached to medical wastes and transmitting information related to the medical wastes including disposal location, date of generation, disposal weight, and date of disposal to the server.

12. The medical waste sterilizer of claim 10,
further comprising a barcode reader connected to a server, which is capable of reading a barcode attached to medical wastes and transmitting information related to the medical wastes including disposal location, date of generation, disposal weight, and date of disposal to the server.

13. A method for operating a medical waste sterilization comprising:
shredding the medical wastes that require a sterilization operation;
spraying acidic sterilizing solution or water onto the shredded medical wastes; and
using two sterilization vessels for a sterilization operation for the medical wastes sprayed with the sterilizing solution and for a waste disposal operation after the sterilization operation in a sequential manner, performing a sterilization operation of medical wastes is in one sterilization vessel while a waste disposal operation is simultaneously performed in the other sterilization vessel.
